(19) United States
(12) Patent Application Publication
(10) Pub. No.: US 2023/0023159 A1
(43) Pub. Date: Jan. 26, 2023

(54) AUGMENTING ELECTRONIC HEALTH RECORDS SYSTEMS

(71) Applicant: Novo Health Inc., Lehi, UT (US)

(72) Inventors: David Brodegard, Lehi, UT (US); Scott Crunkleton, Lehi, UT (US); Robert Taylor, Highland, UT (US)

(21) Appl. No.: 17/874,190

(22) Filed: Jul. 26, 2022

Related U.S. Application Data
(60) Provisional application No. 63/225,897, filed on Jul. 26, 2021.

(51) Int. Cl. G06F 16/958; G16H 10/65; G06F 16/957

(52) U.S. Cl. CPC G06F 16/958; G16H 10/65; G06F 16/9577

(57) ABSTRACT

Techniques for augmenting electronic health records systems by integrating third-party applets via a web browser extension are described. The web browser extension executes in the context of web pages provided by an electronic health records (“EHR”) management system. The extension obtains patient (and other) data from the underlying web page. Data is obtained at least in part by accessing the Document Object Model (DOM) that represents the current web page provided by the EHR system. The obtained data is then provided by the extension to one or more third-party applets, which provide extended functionality relevant to a given medical practice. For example, one applet may provide remote patient monitoring. Another example applet may provide predictive diagnostics, warnings, or the like. The described techniques extend and integrate third-party functionality via applets without asking or forcing the user to context switch out of their EHR every time they desire to access external functionality.

### CROSS-REFERENCE TO RELATED APPLICATION

This application claims the benefit of U.S. Provisional Patent Application No. 63/225,897, entitled “AUGMENTING ELECTRONIC HEALTH RECORDS SYSTEMS,” filed Jul. 26, 2021, the entire content of which is incorporated herein by reference in its entirety.

### TECHNICAL FIELD

The present disclosure relates to methods, techniques, and systems for augmenting electronic health records systems by integrating third-party applets via a web browser extension.

### BACKGROUND

Electronic health records (“EHR”) systems are used by healthcare providers, including doctors, nurses, therapists, and the like, in order to track and manage the care of their patients. While EHR systems typically offer baseline functionality for tracking patient information and related data, further functionality may be limited. For example, an EHR system may not provide modern mechanisms for communicating with patients, such as video chat. As another example, an EHR system may not provide a mechanism for tracking the time that a caregiver has spent with a particular patient. Adding such features to an existing EHR system requires convincing the system developer to add the features, which is often a time-consuming and expensive process. The above issues are compounded by the fact that many providers use multiple distinct EHR systems, which may provide inconsistent and/or varying levels of functionality.

### SUMMARY

A first embodiment provides a process for integrating third-party functionality with an electronic health record system, comprising: providing a web browser extension, configured to provide access to third-party applets in the context of a web page provided by the electronic health record (EHR) system by accessing a Document Object Model (DOM) data structure managed by a web browser, wherein the DOM data structure represents the web page; selecting, based on the EHR system, an adapter library that defines functions for reading and modifying the DOM data structure; selecting one or more applets based on a user and/or organization associated with the EHR system; executing the one or more applets; processing the DOM to identify a data item presented in the web browser; and providing the identified data item to the one or more applets for processing.

Another embodiment extends one or more of the above processes by further receiving a notification that the user has logged into the EHR system; automatically logging the user into an authentication system associated with the web browser extension; and selecting one or more applets based on the identity of the user. Another embodiment extends one or more of the above processes by further tracking time the user has spent with a third-party applet; and entering the tracked time into a billing system associated with the EHR system. Another embodiment extends one or more of the above processes by further processing the DOM to detect a patient identifier; and providing the patient identifier to each of the one or more apps. A further embodiment extends one or more of the above processes by further processing the DOM to detect a patient identifier; and creating a new patient record for use with one of the apps. Another embodiment extends one or more of the above processes by further receiving a notification from an applet; and displaying an indicator that directs the user to provide attention to the applet. A further embodiment extends one or more of the above processes by further obtaining data from the web page provided by the EHR system; and synchronizing the obtained data with a second EHR system.

Other embodiments provide computing systems and/or computer-readable media that store instructions that are configured to execute one or more of the above processes.

### BRIEF DESCRIPTION OF THE DRAWINGS

FIG. 1 is a block diagram of a web browser extension according to an example embodiment.

FIG. 2 is a sequence diagram according to an example embodiment.

FIGS. 3A-3C are flow diagrams illustrating processes performed by example embodiments.

FIGS. 4A-4G are screenshots of a web browser extension and applets according to an example embodiment.

FIG. 5 is a block diagram of a computing system for implementing a browser extension according to an example embodiment.

### DETAILED DESCRIPTION

Embodiments described herein provide methods, devices, and systems for augmenting electronic health records systems by integrating third-party applets via a web browser extension. The web browser extension executes in the context of web pages provided by an electronic health records (“EHR”) management system, such as one that is typically used in a hospital, clinic, or doctor's office. The extension obtains patient (and other) data from the underlying web page. Data is obtained at least in part by accessing the Document Object Model (DOM) that represents the current web page provided by the EHR system.

The obtained data is then provided by the extension to one or more third-party applets, which provide extended functionality relevant to a given medical practice. For example, one applet may provide remote patient monitoring. Another example applet may provide predictive diagnostics, warnings, or the like. The described techniques extend and integrate third-party functionality via applets without asking or forcing the user to context switch out of their EHR every time they want to access external functionality.

Some embodiments provide automatic authentication. When user is logged into their EHR, the extension automatically detects the user and logs them into the extension's authentication service. This enables seamless access to applets associated with the logged in user and/or their organization. The extensions authentication service is typically hosted by a remote computing system, sometimes referred to herein as an “extension support computing system.”

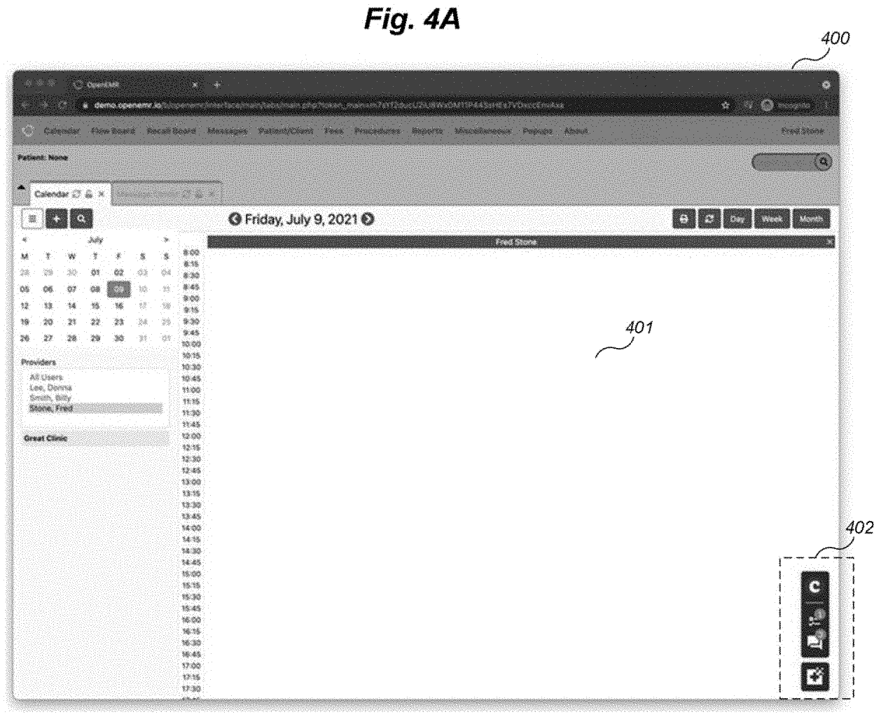

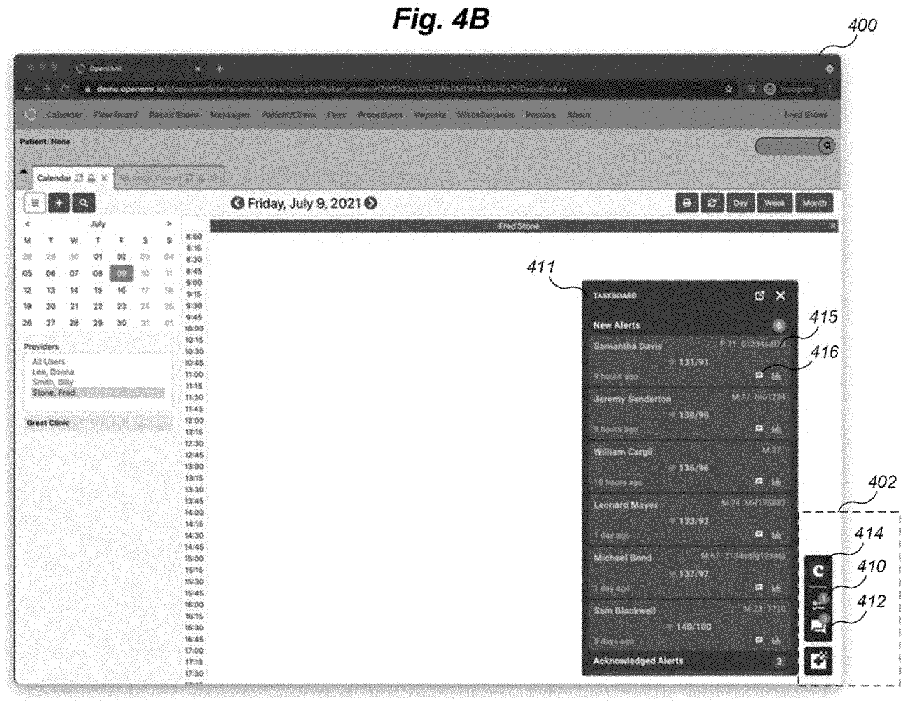

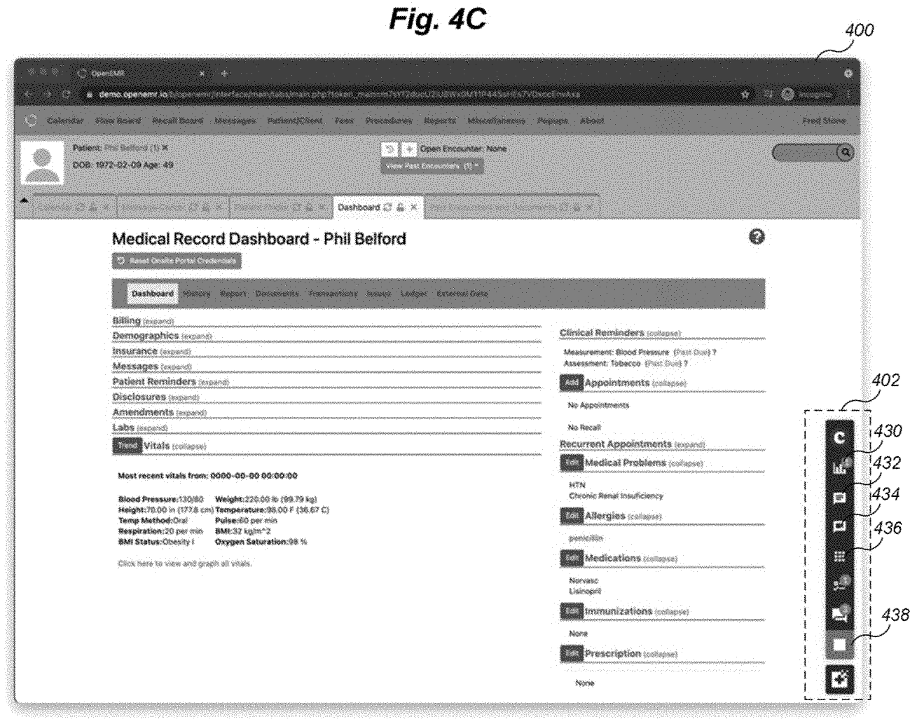

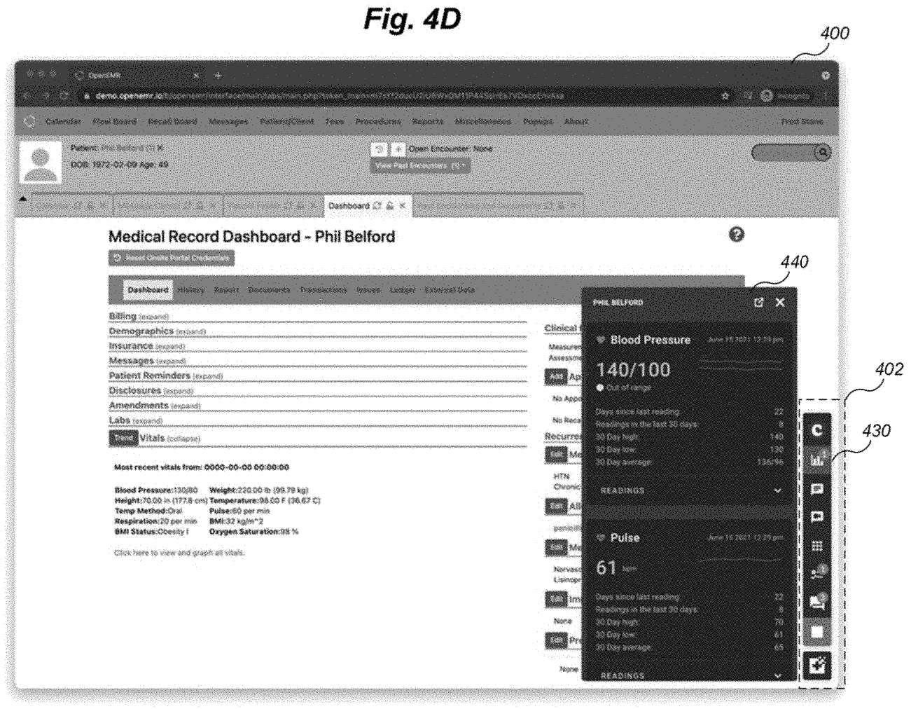

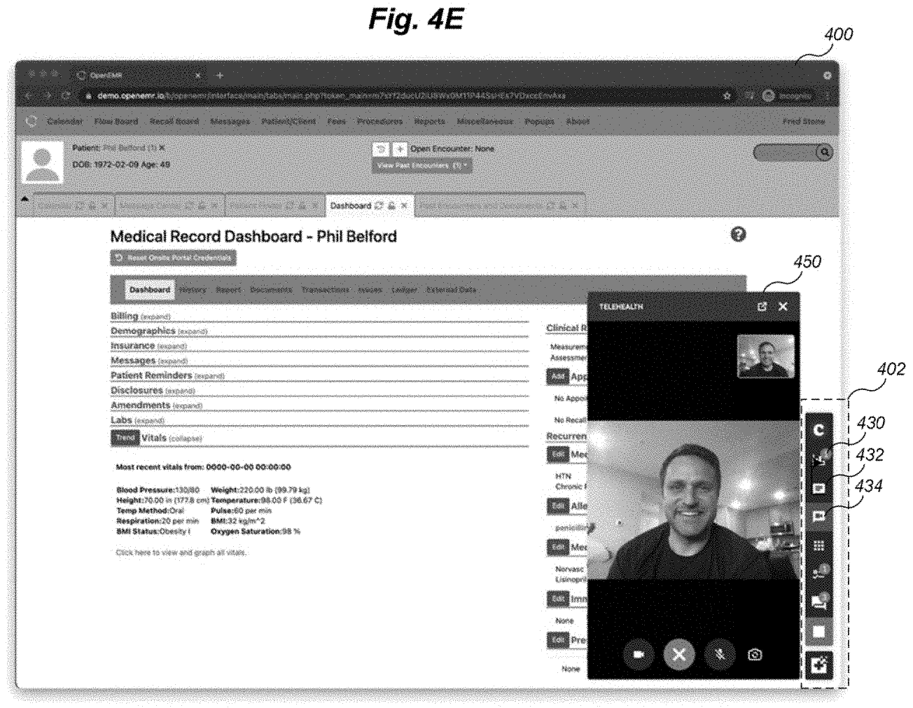

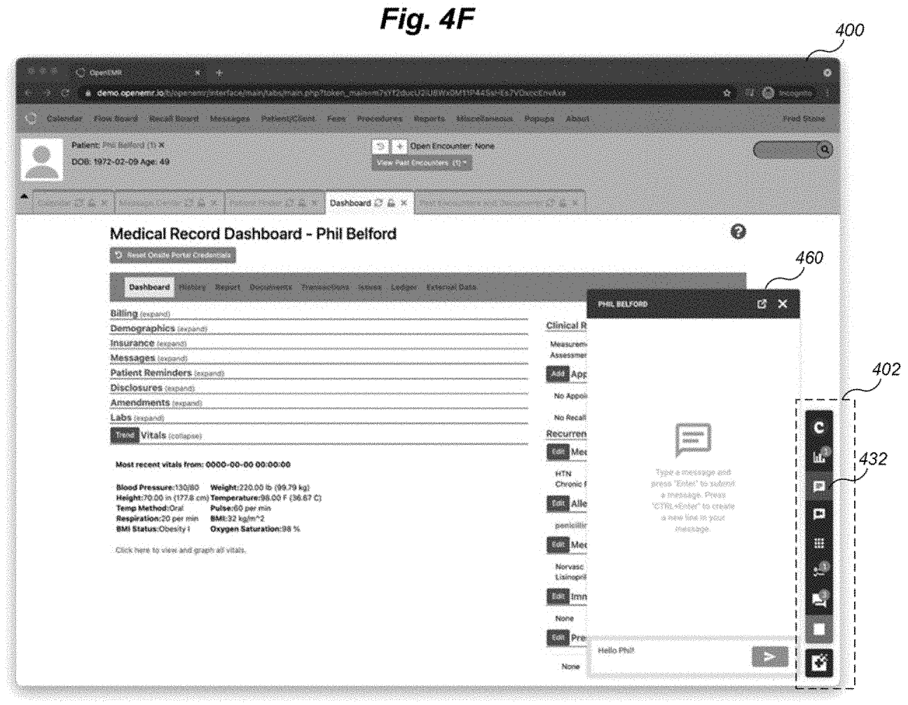

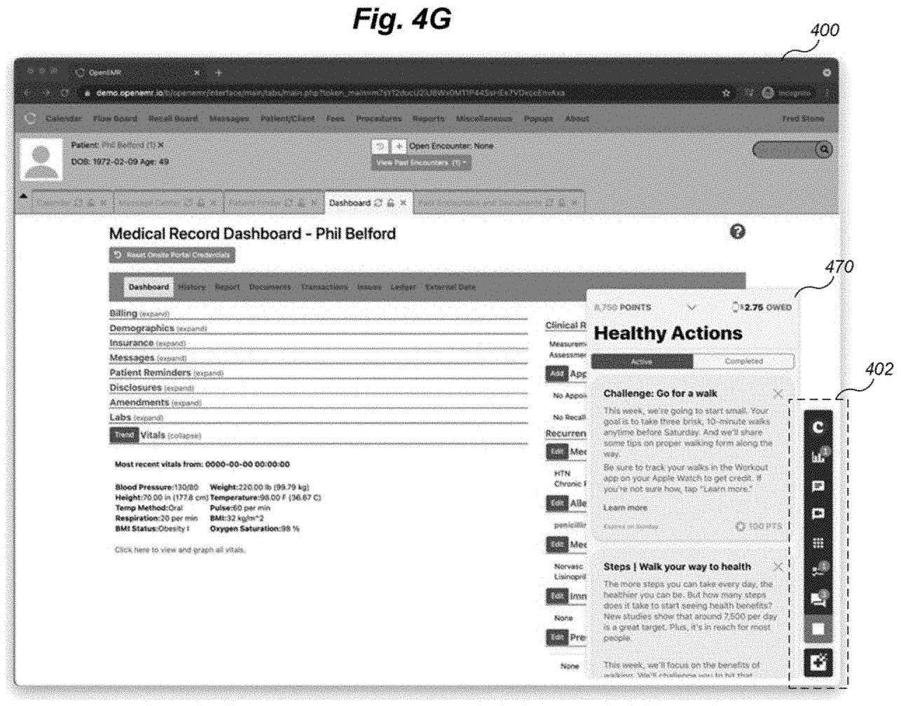

The extension automatically identifies and surfaces applets based on one or more factors, including patient identifiers, user identifiers, organization, and the like. Thus, when a user logs into their EHR, the extension detects the user login, and finds applets that are associated with the user or their organization. These applets may be loaded from the extension support computing system. Screenshots of a web browser extension and example applets are shown and described with reference to FIGS. 4A-4G, below.

The extension includes multiple novel user interface features to facilitate a seamless and efficient experience for the user. The extension executes and displays itself in the context of the web page without being overly intrusive to the underlying interaction with the EHR. To preserve screen real estate, the extension stacks icons to represent relevant applets. The extension also badges its home button with indicators of notifications from applets: applets automatically determine (based on data received from the extension or other sources) whether they need attention and notify the extension; in response, the extension adds an indicator to its home button to notify user of this fact. The indicator may be an icon, number of the like. For example, if the app is a messaging app, the indicator may be the number of new, unread messages.

The extension may also support a multi-view applet interface, including a first “integrated” view that exists in the EHR page and a second “Applet” view that exists separate from the EHR page.

The extension may also support patient discovery and automatic applet enrollment, including pre-population of new patient based on predefined patient lists, EHR data, and/or API calls to back-end records system. In some embodiments, the extension manages a list of patients that is parallel to (or a subset of) the patients that are represented in a given EHR system. When new patients are created in the EHR system, the extension automatically creates a corresponding patient record for its own purposes, possibly saving that record to the extension support computing system.

The extension may also be configured to integrate time keeping functionality across the EHR and one or more applets. The extension can capture time spent in one or more applets and the EHR, and then facilitate the time entry into a billing system associated with the EHR and/or the extension support computing system.

The extension may also synchronize data between two EHR systems. The extension can read data from a first EHR system and transfer or synchronize with a second EHR system. For example, the extension may transmit updated patient data from one EHR system to another. In some embodiments, EHR systems provide APIs that can be utilized to upload data into the system.

In some embodiments, the extension provides access to a hosted applet marketplace, where the provider of the extension and other parties can offer applets that integrate with one or more EHR systems.

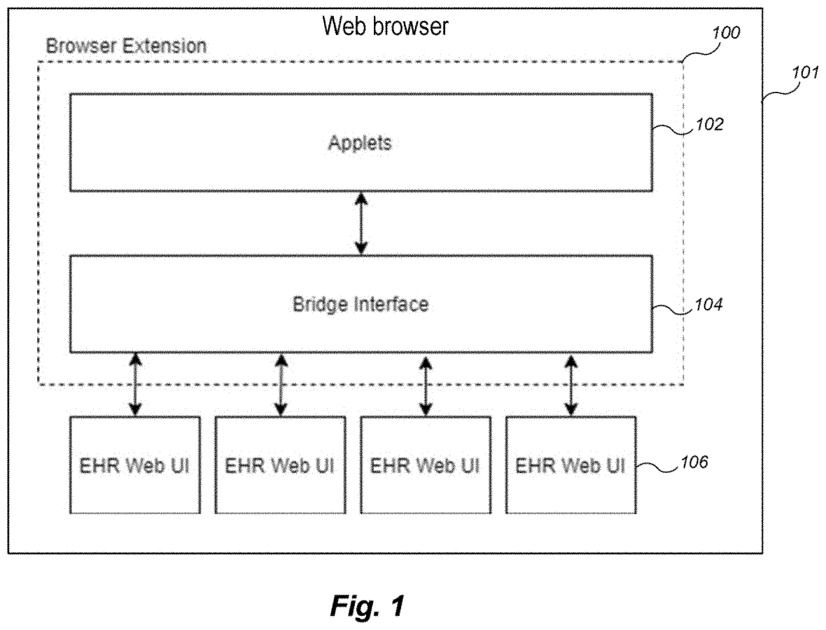

FIG. 1 is a block diagram of a web browser extension 100 according to an example embodiment. In FIG. 1, the extension 100 is shown executing within a web browser 101. The extension 100 includes one or more applets 102 communicating with a bridge interface 104. The bridge interface 104 acts as an intermediary between the applets 102 and one or more EHR Web user interfaces 106 provided by web pages rendered by the browser. The extension may also integrate with other services or systems that are not shown, including the extension support computing system (e.g., hosting an authentication service, applet storage, and the like) and/or an applet marketplace that hosts third-party apps.

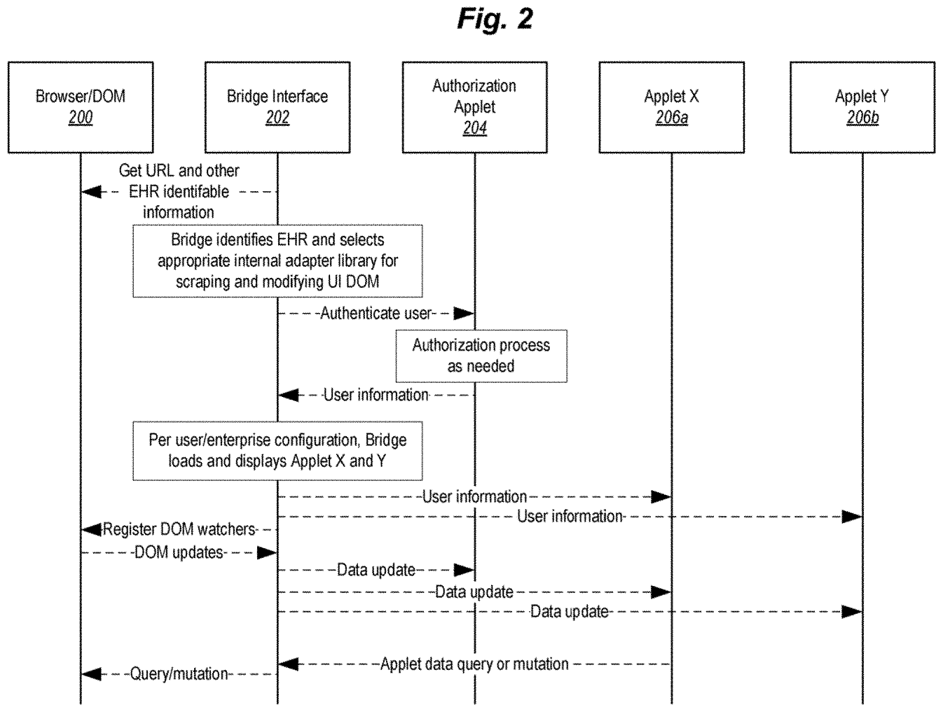

FIG. 2 is a sequence diagram according to an example embodiment. FIG. 2 shows timing and communication flow between a browser/DOM 200, a bridge interface 202, an authentication applet 204, and two third-party applets X (206a) and Y (206b). In the sequence of FIG. 2, the browser 200 initially receives a URL and possibly other information that can be used to identify the EHR providing the web page. Based on the URL and other identifying information, the bridge interface 202 identifies the EHR and selects appropriate internal adapter library for scraping and modifying a document object model that is used to represent a user interface displayed by the Web browser.

The bridge interface 202 then instructs the authorization applet to authenticate the user. In response, the authorization applet 204 executes an authentication process and if successful, provides user information to the bridge interface 202. The authorization applet 204 interacts with an authentication service associated with the extension/bridge. As noted, this service may be hosted by the extension support computing system. This service manages users and organizations that utilize the bridge and is independent of any other EHR authentication service. Once the user is authenticated, the bridge interface 202 loads and displays one or more applets, such as applet X and applet Y, based on the user identity and/or the user's organization. The bridge interface 202 then transmits user information to applets X and Y, which may make use of that information to customize their appearance and/or function.

The bridge interface 202 also registers DOM watchers with the web browser 200. A DOM watcher causes the web browser 200 to notify the bridge interface whenever a specified change or update to the DOM occurs. For example, if a user clicks a particular element of the DOM or a particular health data value (e.g., blood pressure) is updated, a watcher may notify the bridge interface 202 of the event/change. The bridge interface 202 in response may process the DOM to retrieve additional information (e.g., finding a patient identifier, locating a patient health data value). The bridge interface 202 then provides the data from the DOM update and/or determined by the bridge interface 202 to the applets 204 and 206.

The applets 204 and 206 interact with or query the DOM via an interface provided by the bridge interface 202. When one of the applets 204 or 206 make a request to query or modify the DOM, the bridge interface 202 responds by performing the necessary query and/or mutation operation on the DOM.

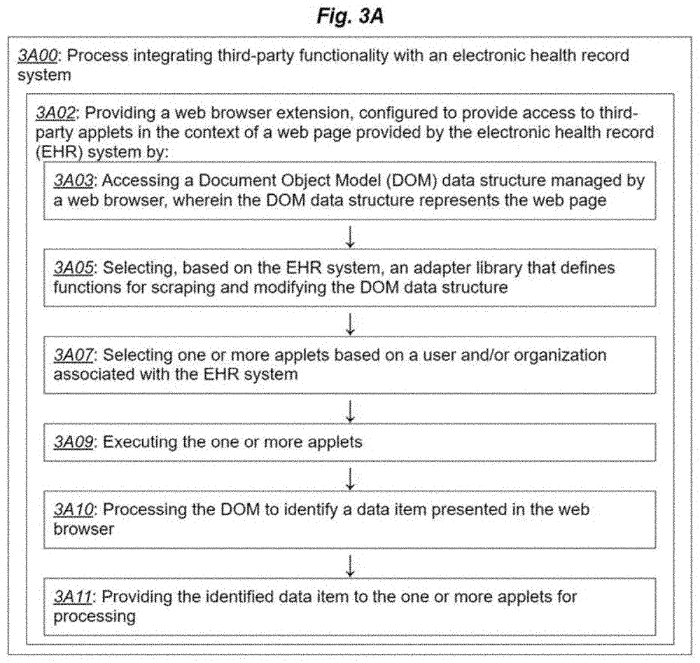

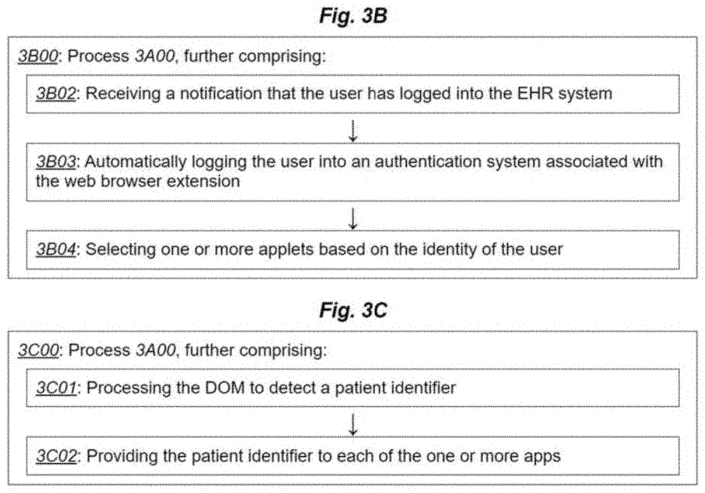

FIG. 3A is a flow diagram of example logic for integrating third-party functionality with an EHR system. This process is typically performed by a web browser extension that executes in the context of a web-based application that is provided by an EHR system. The extension obtains patient data from a web UI instantiated by a web page provided by the EHR system and uses that information to access and integrate with one or more applets, such as a Remote Patient Monitoring (“RPM”) application. Applets offer extended functionality that is relevant to a given medical practice. FIG. 3A illustrates a process 3A00 that includes block(s) 3A02 described below.

Block 3A02 includes providing a web browser extension, configured to provide access to third-party applets in the context of a web page provided by the electronic health record (EHR) system by: performing block(s) 3A03-3A11 described below.

Block 3A03 includes accessing a Document Object Model (DOM) data structure managed by a web browser, wherein the DOM data structure represents the web page. The extension, once added to the user's browser, receives access to the underling DOM data structure of the web page.

Block 3A05 includes selecting, based on the EHR system, an adapter library that defines functions for accessing and modifying the DOM data structure. The process can access and load a set of custom functions that are specialized to read and modify the DOM data structure provided by the specific EHR system.

Block 3A07 includes selecting one or more applets based on a user and/or organization associated with the EHR system. The process uses the identity of the user and/or their organization to load a custom set of applets that are associated with the user.

Block 3A09 includes executing the one or more applets.

Block 3A10 includes processing the DOM to identify a data item presented in the web browser. In some cases this processing may be initiated in response to an event or notification provided by the browser, such as that the DOM has been updated or otherwise modified. The data item may be, for example, a patient identifier (e.g., name, birthday, address), medical data related to the patient (e.g., heart rate, blood pressure, test results), communications (e.g., messages), calendar data, or the like.

Block 3A11 includes providing the identified data item to the one or more applets for processing. Each applet adheres to a standard interface to receive data from the extension. The applet is also able to respond by requesting execution of a query or modification of the DOM data structure.

FIG. 3B is a block diagram of example logic that extends the module 3A00 of FIG. 3A. The extension process provides a seamless authentication experience for the user by detecting the user's log into their EHR system, and then automatically logging the user into an authentication system that is associated with the extension, such that the extension can access user-specific settings, preferences, applet choices, and the like. More specifically, FIG. 3B illustrates a process 3600 and which further includes the following block(s).

Block 3602 includes receiving a notification that the user has logged into the EHR system. This notification may be received by watching the DOM for changes that reflect successful log in, such as the appearance of a message, an EHR home screen, or the like.

Block 3603 includes automatically logging the user into an authentication system associated with the web browser extension. The extension process here logs into a support system that is separate from the EHR system. The support system manages organizations, groups, and users that operate the extension. The support system provides a host for organizational data (e.g., users, groups, permissions) and applets.

Block 3604 includes selecting one or more applets based on the identity of the user. A user (or their parent organization) may have previously selected one or more applets that should be made visible upon login.

FIG. 3C is a block diagram of example logic that extends the module 3A00 of FIG. 3A. More specifically, FIG. 3C illustrates a process 3C00 and which further includes the following block(s).

Block 3C01 includes processing the DOM to detect a patient identifier. In typical embodiments, the process registers watchers or event handlers that are notified of changes to the DOM. For example, when a DOM is updated (e.g., because the user has clicked on a patient record), this process is notified and then processes the DOM to locate a patient identifier.

Block 3C02 includes providing the patient identifier to each of the one or more apps. Once the patient identifier has been identified, the web browser extension passes is to the apps, which take an appropriate action in response.

FIGS. 4A-4G are screenshots of a web browser extension and applets according to an example embodiment. FIG. 4A shows a web browser 400 displaying a web page 401 provided by the EHR system. The web page 401 presents a user interface that is configured to be operated by a user to interact with the EHR system. In FIG. 4A, the inventive web browser extension 402 (sometimes referred to as “Bridge”) is injected on top of the web page 401 and initially only takes up a small space in the lower right. The overlay of the extension 402 is designed to be minimally invasive and can be docked to any of the four corners of the EHR user interface. This screenshot shows Bridge as a user would see it immediately on logging in to the EHR. No patient specific context means that only the to-do list (taskboard) and the messaging inbox are shown. Clicking the Bridge icon will toggle the expansion and contraction of the toolbar above.

Note that the presentation of the bridge extension and corresponding apps may occur automatically upon login to the EHR system. The bridge extension detects the EHR login (e.g., by monitoring the DOM for changes that reflect the successful login) and then automatically logs into the extension support computing system, which provides a user-specific set of applets for display in the extension. For example, if the bridge detects the loading of a specific page (e.g., a successful login page) or the presence of a particular DOM element (e.g., a message reflecting successful login, the presence of a user identifier), the bridge will automatically log into the extension support computing system, using credentials associated with the identified user. The bridge and/or the extensions support computing system retains a mapping between EHR user identifiers and bridge users. A given bridge user may have multiple distinct EHR user identifiers, such as may be associated with distinct EHR systems.

In FIG. 4B, from a non-patient screen in the EHR a user can easily select a taskboard control 410 which displays a taskboard 411 showing patient alerts for the entire patient population. From this taskboard 411 the user can easily view the latest values for every patient. From this same screen a user can text directly with any patient in the population, for example by selecting a texting control 416 contained within a particular patient alert 415. Notice that in addition to the taskboard control 410, and the conversations inbox 412, third party apps (like the clover assistant 414) that the organization has chosen to include are also available to the user.

In FIG. 4C, Bridge 402 reacts to navigation within the EHR and displays additional functionality based on the patient context. Note that the number of controls displayed by the bridge 402 has increased as compared to the screenshot of FIG. 4B. In the illustrated example, Phil Belford is an enrolled patient, so Bridge displays controls for selecting applets for the most recent data 430, messaging 432, video calls 434, as well as third party apps 436. The green button 438 indicates that time is being tracked. The time can be started and stopped by clicking the green button 438.

The extension can track time that the user spends in each applet, by detecting when an applet is opened or closed. Alternatively, the extension may track time on a per-patient basis. Thus, time tracking begins when a specific patient record is accessed in the underling EHR. Time tracking for that patient ends when a new patient is accessed, or when the current patient's record is closed, or similar condition. In some cases, time tracking may also consider user activity. Thus, if a user does not interact with the EHR for a specified amount of time, the bridge may end time tracking for the current patient. Tracked time can then be automatically transmitted to a remote time keeping system or service. In some embodiments, the time keeping service is associated with the EHR system. In other embodiments, the time keeping service is provided by the same computing system that hosts the browser extension and applets.

In FIG. 4D, selecting the “latest values” applet control 430 will display a latest values applet 440 that shows the most recent readings from any and all connected devices. From this applet 440 the user can view the graph and list view of all of the latest information. The user is able to copy values for pasting into the EHR as well as export a patient summary (e.g., as PDF or other format) for upload into the EHR.

In FIG. 4E, from within the patient chart inside of the EHR the user can initiate a video call 450 directly with the patient by selecting the video call control 434. The user can easily toggle between the video applet 450 and messaging 460 (FIG. 4F) or latest values applet 440 by selecting the corresponding control 430, 432, or 434.

In FIG. 4F, the messaging applet 460 allows the user to send and receive SMS messages directly with the patient. By selecting the control 432, the extension displays the messaging applet 460.

As shown in FIG. 4G, Bridge is not limited to Bridge-created applets. A third-party app 470 is shown in FIG. 4G. Third-party apps are granted the same permissions and access to the EHR content that Bridge applets are given. The underlying software does all of the heavy lifting of parsing the DOM to extract the pertinent data and the data is then passed to the applets. Third party software can be easily integrated through the Bridge SDK (software development kit) and corresponding API (application program interface) and typically can be fully integrated with minimal effort. In some embodiments, third-party apps are not hosted by Bridge but are made available to the end user through the Bridge Marketplace. The Bridge Marketplace is similar to app stores in the sense that organization administrators can chose which applets they would like to include for their organization and the selected applets are immediately made available to all users.

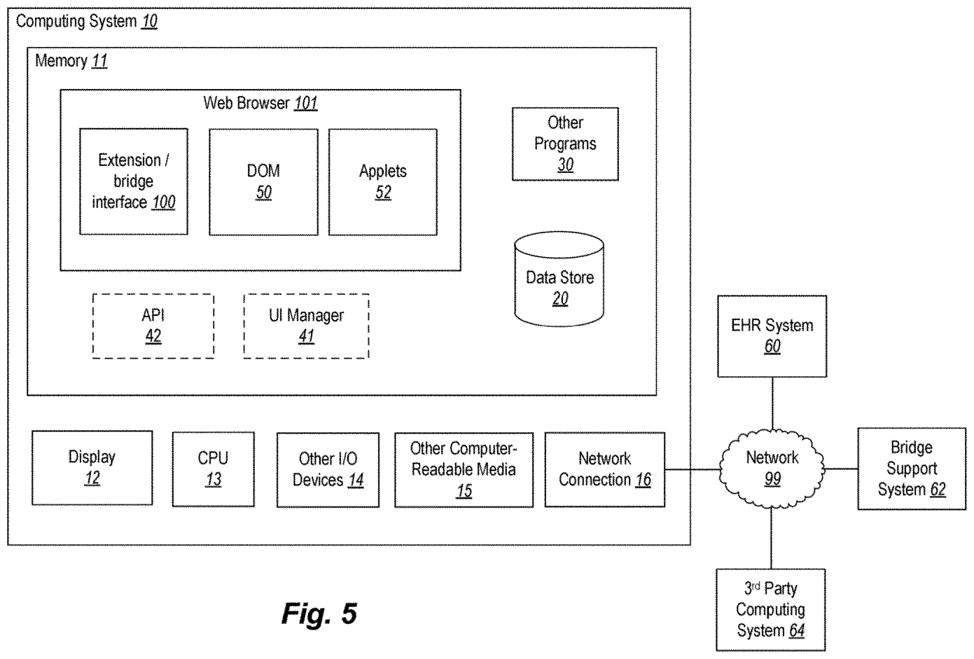

FIG. 5 is a block diagram of a computing system for implementing a browser extension according to an example embodiment. In particular, FIG. 5 shows a computing system 10 that executes the browser extension 100 described herein. Similar techniques can be applied to implementing other computing systems illustrated in FIG. 1 and elsewhere herein.

The computing system 10 may comprise one or more computing systems and may span distributed locations. In addition, each block shown may represent one or more such blocks as appropriate to a specific embodiment or may be combined with other blocks. Moreover, the various blocks may physically reside on one or more machines, which use standard remote procedure call (e.g., RPC) or proprietary inter-process communication mechanisms (IPC) to communicate with each other.

In the embodiment shown, computing system 10 comprises a computer memory (“memory”) 11, a display 12, one or more Central Processing Units (“CPU”) 13, Input/Output devices 14 (e.g., keyboard, mouse, CRT or LCD display, and the like), other computer-readable media 15, and a network connection 16. The browser extension 100 is shown executing within a Web browser 101 residing in memory 11. The Web browser 101 also includes a DOM 50 and one or more applets 52. The DOM 50 represents a web page that is currently being displayed by the browser 101. The applets 52 represent code modules that execute within the Web browser and that interact with the extension 100 to obtain information about and/or modify the DOM 50. The applets are not necessarily “Java Applets.” An applet may be any module of code (e.g., written in JavaScript, TypeScript, Java, or the like) that executes within the context of the web browser. In Applets are typically stored and loaded independently of a particular web page. An applet typically provides some function related to or in extension of a Web page or service that is currently being displayed or interacted with via a Web browser.

In other embodiments, some portion of the contents, some or all of the components of the extension 100 may be stored on and/or transmitted over the other computer-readable media 15. The extension 100 preferably executes on one or more CPUs 13 and performs the techniques described herein. Other code or programs 30 (e.g., an administrative interface, a Web server, and the like) and potentially other data repositories, such as data repository 20, also reside in the memory 11, and preferably execute on one or more CPUs 13. Of note, one or more of the components in FIG. 5 may not be present in any specific implementation. For example, some embodiments may not provide other computer readable media 15 or a display 12.

The extension 100 is shown executing in the memory 11 of the system 10. Also included in the memory 11 are a user interface manager 41 and an application program interface (“API”) 42. The user interface manager 41 and the API 42 are drawn in dashed lines to indicate that in other embodiments, functions performed by one or more of these components may be performed externally to the extension 100.

The UI manager 41 provides a view and a controller that facilitate user interaction with the system 140 and its various components. For example, the UI manager 41 may provide interactive access to the extension 100, such that users or administrators can interact with the extension 100 such as to upload or manage test lists, enter new patient data, or the like. In some embodiments, access to the functionality of the UI manager 41 may be provided via a Web server, possibly executing as one of the other programs 30. In such embodiments, a user operating a Web browser executing on a client system or device can interact with the module 100 via the UI manager 41.

The API 42 provides programmatic access to one or more functions of the extension 100. For example, the API 42 may provide an interface that can be used by the applets 52 to interact with the DOM 50. As another example, the API 42 may provide a programmatic interface to one or more functions of the extension 100 that may be invoked by one of the other programs 30 or some other module. In this manner, the API 42 facilitates the development of third-party software, such as user interfaces, plug-ins, adapters (e.g., for integrating functions of the module 100 into Web applications), and the like. Although the API 42 is drawn external to the browser 101, in other embodiments it may be part of the extension 100.

The extension 100 may interact using network connection 16 via a network 99 with other devices/systems including an EHR system 60, a bridge support computing system 62, and third-party computing system 64. The network 99 may be any combination of media (e.g., twisted pair, coaxial, fiber optic, radio frequency), hardware (e.g., routers, switches, repeaters, transceivers), and protocols (e.g., TCP/IP, UDP, Ethernet, Wi-Fi, WiMAX) that facilitate communication between remotely situated humans and/or devices. Computing systems 60, 62, and 64 may be constituted similarly to system 10.

The bridge support computing system 62 may act as a central, network accessible host for storing, managing, and distributing browser extensions and related functionality. For example, the system 62 may host the bridge and/or one or more applets (e.g., in an app marketplace), such that they may be downloaded and installed into a user's web browser. The system 62 may also manage users, groups, and organizations that utilize the extension 100. For example, Organization X may have a list of users that are authorized to use the extension. To enforce this authorization, users must log in with the system 62 via the extension 100. Once logged in, each user may have specific permissions to install and/or operate one or more applets or other functions of the extension 100.

Note that one or more general purpose or special purpose computing systems/devices may be used to implement and/or execute the extension 100. However, just because it is possible to implement the extension 100 on a general purpose computing system does not mean that the techniques themselves or the operations (taken alone or in combination) required to implement the techniques are conventional or well known. The techniques are not conventional at least because they address and improve an existing technology, such as by improving the operation, integration, or efficiency of one or more computing systems.

In an example embodiment, components/modules of the extension 100 are implemented using software programming techniques. For example, the extension 100 may be implemented as a “native” executable running on the CPU 13, along with one or more static or dynamic libraries. In other embodiments, the extension 100 may be implemented as instructions processed by a virtual machine that executes as one of the other programs 30.

The various components may be implemented using more monolithic programming techniques, for example, as an executable running on a single CPU computer system, or alternatively decomposed using a variety of structuring techniques, including but not limited to, multiprogramming, multithreading, client-server, or peer-to-peer, running on one or more computer systems each having one or more CPUs. Some embodiments may execute concurrently and asynchronously, and communicate using message passing, remote procedure call, or other distributed computing paradigms. Equivalent synchronous embodiments are also supported. Also, other functions could be implemented and/or performed by each component/module, and in different orders, and by different components/modules, yet still achieve the described functions.

In addition, programming interfaces to the data stored as part of the extension 100, such as in the data store 20, can be available by language-specific APIs; libraries for accessing files, databases, or other data repositories; through representational languages such as XML; or through Web servers, FTP servers, or other types of servers providing access to stored data. The data store 20 may be implemented as one or more database systems, file systems, or any other technique for storing such information, or any combination of the above, including implementations using distributed computing techniques.

Furthermore, in some embodiments, some or all of the components of the extension 100 may be implemented or provided in other manners, such as at least partially in firmware and/or hardware, including, but not limited to one or more application-specific integrated circuits (“ASICs”), standard integrated circuits, controllers executing appropriate instructions, and including microcontrollers and/or embedded controllers, field-programmable gate arrays (“FPGAs”), complex programmable logic devices (“CPLDs”), and the like. Some or all of the system components and/or data structures may also be stored as contents (e.g., as executable or other machine-readable software instructions or structured data) on a computer-readable medium (e.g., as a hard disk; a memory; a computer network or cellular wireless network or other data transmission medium; or a portable media article to be read by an appropriate drive or via an appropriate connection, such as a DVD or flash memory device) so as to enable or configure the computer-readable medium and/or one or more associated computing systems or devices to execute or otherwise use or provide the contents to perform at least some of the described techniques. Some or all of the components and/or data structures may be stored on tangible, non-transitory storage mediums. Some or all of the system components and data structures may also be stored as data signals (e.g., by being encoded as part of a carrier wave or included as part of an analog or digital propagated signal) on a variety of computer-readable transmission mediums, which are then transmitted, including across wireless-based and wired/cable-based mediums, and may take a variety of forms (e.g., as part of a single or multiplexed analog signal, or as multiple discrete digital packets or frames). Such computer program products may also take other forms in other embodiments. Accordingly, embodiments of this disclosure may be practiced with other computer system configurations.

All of the U.S. patents, U.S. patent application publications, U.S. patent applications, foreign patents, foreign patent applications and non-patent publications referred to in this specification and/or listed in the Application Data Sheet are incorporated herein by reference, in their entirety.

While embodiments of the invention have been illustrated and described, as noted above, many changes can be made without departing from the spirit and scope of the invention. Accordingly, the scope of the invention is not limited by the above disclosure.

1. A method for integrating third-party functionality with an electronic health record system, comprising:
providing a web browser extension, configured to provide access to third-party applets in the context of a web page provided by the electronic health record (EHR) system by:
accessing a Document Object Model (DOM) data structure managed by a web browser, wherein the DOM data structure represents the web page;
selecting, based on the EHR system, an adapter library that defines functions for reading and modifying the DOM data structure;
selecting one or more applets based on a user and/or organization associated with the EHR system;
executing the one or more applets;
processing the DOM to identify a data item presented in the web browser; and
providing the identified data item to the one or more applets for processing.

2. The method of claim 1, further comprising:
receiving a notification that the user has logged into the EHR system;
automatically logging the user into an authentication system associated with the web browser extension; and
selecting one or more applets based on the identity of the user.

3. The method of claim 1, further comprising:
tracking time the user has spent with a third-party applet; and
entering the tracked time into a billing system associated with the EHR system.

4. The method of claim 1, further comprising:
processing the DOM to detect a patient identifier; and
providing the patient identifier to each of the one or more apps.

5. The method of claim 1, further comprising:
processing the DOM to detect a patient identifier; and
creating a new patient record for use with one of the apps.

6. The method of claim 1, further comprising:
receiving a notification from an applet; and
displaying an indicator that directs the user to provide attention to the applet.

7. The method of claim 1, further comprising:
obtaining data from the web page provided by the EHR system; and
synchronizing the obtained data with a second EHR system.

8. The method of claim 1, further comprising:
integrating a remote patient monitoring applet with the EHR system.

9. A computing system, comprising:
a processor; and
a memory that stores Web browser extension instructions that are configured, when executed by the processor and within a Web browser, to provide access to third-party applets in the context of a web page provided by an electronic health record (EHR) system by:
accessing a Document Object Model (DOM) data structure managed by the web browser, wherein the DOM data structure represents the web page;
selecting, based on the EHR system, an adapter library that defines functions for reading and modifying the DOM data structure;
selecting one or more applets based on a user and/or organization associated with the EHR system;
executing the one or more applets;
processing the DOM to identify a data item presented in the web browser; and
providing the identified data item to the one or more applets for processing.

10. The computing system of claim 9, wherein the browser extension instructions are further configured to:
receive a notification that the user has logged into the EHR system;
automatically log the user into an authentication system associated with the web browser extension; and
select one or more applets based on the identity of the user.

11. The computing system of claim 9, wherein the browser extension instructions are further configured to:
track time the user has spent with a third-party applet; and
enter the tracked time into a billing system associated with the EHR system.

12. The computing system of claim 11 wherein the browser extension tracks time by monitoring the opening and closing of the third-party applet.

13. The computing system of claim 9, wherein the browser extension instructions are further configured to:
processing the DOM to detect a patient identifier; and
providing the patient identifier to each of the one or more apps.

14. The computing system of claim 9, wherein the browser extension instructions are further configured to:
process the DOM to detect a patient identifier; and
create a new patient record for use with one of the apps.

15. The computing system of claim 9, wherein the browser extension instructions are further configured to:
receive a notification from an applet; and
display an indicator that directs the user to provide attention to the applet.

16. The computing system of claim 16, wherein the browser extension displays the indicator that directs the user to provide attention to the applet by annotating a button with an icon or text.

17. The computing system of claim 9, wherein the browser extension instructions are further configured to:
obtain data from the web page provided by the EHR system; and
synchronize the obtained data with a second EHR system.

18. The computing system of claim 17, wherein the browser extension synchronizes the obtained data by transmitting the obtained data to an API hosted by the second EHR system.

19. A computer-readable storage medium that persistently stores instructions that are configured, when executed by the processor and within a Web browser, to provide access to third-party applets in the context of a web page provided by an electronic health record (EHR) system by performing a method comprising:
accessing a Document Object Model (DOM) data structure managed by the web browser, wherein the DOM data structure represents the web page;
selecting, based on the EHR system, an adapter library that defines functions for reading and modifying the DOM data structure;
selecting one or more applets based on a user and/or organization associated with the EHR system;
executing the one or more applets;
processing the DOM to identify a data item presented in the web browser; and
providing the identified data item to the one or more applets for processing.
